(19) United States
(12) Patent Application Publication
(10) Pub. No.: US 2013/0251755 A1
(43) Pub. Date: Sep. 26, 2013

(54) SHAPED BODY WITH COLLAGEN-CONTAINING COMPOSITE MATERIAL FOR INTRODUCTION INTO A BONE DEFECT LOCATION

(71) Applicant: Resorba Wundversrgung GmbH & Co. KG, Numberg (DE)

(72) Inventors: Markus FREY, Nurnberg (DE); Michael Ahlers, Stein (DE); Karl-Heinz Sorg, Neustadt/Donau (DE); Christian Huber, Nurnberg (DE)

(73) Assignee: Resorba Wundversrgung GmbH & Co. KG, Numberg (DE)

(21) Appl. No.: 13/622,149

(22) Filed: Sep. 18, 2012

(30) Foreign Application Priority Data
Sep. 19, 2011 (DE) 10 2011 082 960.1

(51) Int. Cl. A61L 27/44

(52) U.S. Cl. CPC A61L 27/44; A61L 27/446
USPC 424/400; 514/17.2; 424/549; 424/602

(57) ABSTRACT

The shaped body is intended for introduction into an alveolar space or into another bone defect location. A composite material with at least a first material component in the form of a collagen material and a second material component in the form of a bone replacement material is provided for the shaped body. The collagen material forms a matrix, in which the bone replacement material is embedded, distributed non-homogeneously, so that a local collagen proportion based on any desired part volume of 30 mm³ is at least 10% everywhere and a global collagen proportion based on the total volume is in the range between 30% and 95%. The shaped body is compressible. A local bone replacement proportion based, in each case, on a part volume of 30 mm³ has its maximum value at an edge face and decreases inwardly proceeding from the edge face, a BRM zone provided with bone replacement material extending into the interior proceeding from the edge face.

### CROSS-REFERENCES TO RELATED APPLICATIONS

This application claims the priority of German Patent Application, Serial No. 10 2011 082 960.1, filed Sep. 19, 2011, pursuant to 35 U.S.C. 119(a)-(d), the content of which is incorporated herein by reference in its entirety as if fully set forth herein.

### FIELD OF THE INVENTION

The invention relates to a shaped body for introduction into an alveolar space or into another bone defect location.

### BACKGROUND OF THE INVENTION

During the medical treatment (relatively small) iatrogenic bone defects may occur. An example of this is the wound cavity after the clearing away of a bone cyst. The tooth pocket (=alveolus) present after a tooth extraction can also be taken to mean a special case of a bone defect of this type.

Various treatments methods are known in dentistry after a tooth extraction. In a first approach, a healing of the alveolus firstly takes place in order to then start again with a dental prosthesis treatment for, for example, a tooth implantation. An undesired shrinkage of bone substance may occur here during the healing period. The cause of this is, for example, an inflammation in the alveolus, which increases the fibrinolytic activity of the blood, which is why no stable blood clot (=coagulum) can form. A disturbed wound healing and a loss of bone tissue occurs. As a consequence of this, the dental prosthesis can either not anchor in an optimal manner or a complex regeneration treatment of the jaw bone even has to be interposed.

In a different approach, directly after the tooth extraction, an implant is inserted. An example of an implant of this type is, for example, described in DE 196 30 034 A1. It contains a hard titanium core, which is inter alia provided with a collagen-containing coating and, after an ingrowth phase, serves as a base for a tooth crown. Problems with the ingrowth can occur in these directly inserted implants, for example because of inflammations in the alveolus.

An implant mount, which is produced for each patient individually in accordance with the size of the alveolus, is furthermore known from DE 10 2006 047 054 A1. A comparatively high production and cost outlay results because of the special individual production. The implant mount consists of ceramic, preferably of the synthetic bone replacement material hydroxylapatite, and has an inwardly decreasing density or increasing porosity. It has a very rigid support structure and after growing together with the jaw bone, serves as a direct base, in which the tooth implant is anchored. An ingrowth of the natural bone into the tooth implant is made more difficult and slowed down when using the implant mount. This implant mount is a permanent avital bone replacement. It is fastened to the jaw by means of screws for better fixing. This implant mount rests very closely on the alveolar wall and can lead to additional injuries there during insertion, so inflammations and delayed wound healing may occur.

Moreover, a resorbable composite body intended for insertion in the alveolus and made of a base body and a covering membrane is known from DE 10 2008 010 893 B3 and from EP 2 249 739 B1. The base body may consist of a collagen material, in which a core made of bone replacement material is completely embedded or in which bone replacement material is incorporated, distributed homogeneously. The composite body is used, for example, after a tooth extraction for bone regeneration and to maintain the existing bone substance, in order to later, after the wound healing has taken place, be able to anchor a tooth implant in the jaw bone. The bone replacement material is relatively hard and inflexible, so additional injuries with the consequences mentioned above may also occur during insertion of the composite body into the alveolus.

A composition for treating one and cartilage defects is also described in DE 10 2007 012 276 A1. The composition contains at least a collagen, which has at least one osteoinductive or chondroinductive active ingredient, and at least one additive of a differentiating and/or growth factor. Furthermore, the composition may optionally contain a filling material. The filling material may be taken to mean a bone replacement material. Type 1 collagen, extracts of native bone, and ceramic materials, such as, for example, tricalcium phosphate and hydroxylapatite are mentioned inter alia as filling materials. The composition may be present as a substantially dimensionally stable body. No details are given about the distribution of the filling material within the composition.

### SUMMARY OF THE INVENTION

An object of the invention is to disclose a shaped body of the type designated at the outset, which brings about improved wound healing and improved bone regeneration and preservation.

A shaped body for introduction into an alveolar space or into another bone defect location, wherein a composite material with at least a first material component in the form of a collagen material and a second material component in the form of a bone replacement material is provided for the shaped body, the collagen material forms a matrix, in which the bone replacement material is embedded, distributed non-homogeneously, so that a local collagen proportion based on any desired part volume of 30 mm³ is at least 10% everywhere and a global collagen proportion based on the total volume is in the range between 30% and 95%, the shaped body is compressible, and a local bone replacement material proportion based on a part volume of 30 mm³ has its maximum value at an edge face and decreases inwardly proceeding from the edge face, a BRM zone provided with bone replacement material extending into the interior proceeding from the edge face, is disclosed to achieve this object.

The shaped body according to the invention is intended for insertion in a bone defect location, which may be located, in particular, in the face region, preferably in the mouth or jaw region. In configurations, in which an introduction into an alveolar space is provided, the shaped body may, in particular, also be called an alveolar filling body or dental filling body.

The shaped body consisting, in particular, of the collagen-containing composite material, by its combined structure of collagen and bone replacement material, assists the natural bone regeneration. The collagen serves as a regenerative guide track and the bone material as a stabilizing component, it being possible for the two components to cooperate synergistically. When being used as an alveolar filling body, the shaped body according to the invention, in contrast to the implant mount according to DE 10 2006 047 054 A1, is not a primary base for anchoring an implant, but rather serves firstly to build up a suitable implant base in advance of a tooth implantation by at least partial regeneration of the natural, physiological bone matrix. An implant inserted later then grows into a vital, physiological bone base. An avital, synthetic base like the implant mount according to DE 10 2006 047 054 A1, on the other hand, does not exhibit any comparable favorable properties for the ingrowth of the implant.

If, in another application, an implant is to be inserted directly into the existing jaw bone, cavities remaining around the implant can also be filled with at least one shaped body according to the invention in the same treatment sitting, in which the implant is also inserted. The treatment duration is thus shortened. The shaped body/bodies inserted in the cavities promote the wound healing and the ingrowth of the implant.

The bone replacement material, after introduction, leads to a relatively rapid stabilization of the bone defect. Complete bone regeneration is, however, only achieved after the comparatively slow resorption of the bone replacement material. Too much introduced bone replacement material therefore stresses the organism, prevents vascularisation and delays the healing process. Because of the non-homogeneous distribution of the bone replacement material (=BRM) within the collagen matrix, the stress from the bone replacement material can be reduced, without dispensing with its positive mechanically stabilizing effects, in particular at the locations at which a stabilization of this type is required. However, on the other hand, there are also regions within the bone defect, in which no support is necessary, for example on the alveolar base. The use of the bone replacement material can be metered as required and adapted to the purpose of application by means of the non-homogeneous distribution of the bone replacement material within the collagen matrix. The quantity of the bone replacement material applied can therefore be reduced to the necessary amount. There are zones within the shaped body with little or no bone replacement material at all. The shaped body can therefore also contain, in particular, at least one bone replacement material-free zone. The latter may certainly be very small and, for example, only make up about 10% of the total volume of the shaped body. In particular, the percentage proportion of the bone replacement material-free zone based on the total volume is in the range from 30% to 90%, preferably from 40% to 85%, and preferably from 50% to 75%.

However, a configuration is basically also possible, in which a certain pro-portion of bone replacement material, even if differing from one another locally, is provided everywhere in the shaped body. Thus, for example, a fluent or continuous course of the proportion of incorporated bone replacement material may be provided, without a local bone replacement material proportion completely becoming zero anywhere within the shaped body.

The global collagen proportion is, in particular, in each case, between 55% and 95%, preferably between 60% and 90%, and preferably between 70% and 80%.

The collagen also has a favorable effect on the bone regeneration. In contrast to the bone replacement material, collagen is a soft, plastically deformable material. It contributes less to the mechanical stabilization. Instead, because of its very easy resorbability, it impairs the natural bone regeneration (osteogenesis and vascularisation) significantly less than the bone replacement material.

With the combined structure of the shaped body made of collagen and bone replacement material, the advantages of both materials can be utilized. In a preferred embodiment, a composite material, which consists exclusively of the collagen material and the bone replacement material, is provided for the shaped body. Other embodiments with additional materials and/or substances within the composite material are, however, basically also possible.

The shaped body is advantageously compressible overall. In particular, the shaped body can be compressed significantly better than comparable configurations known hitherto, such as, for example, the implant mount according to DE 10 2006 047 054 A1, in which practically no compression is possible. The non-homogeneous embedding of the bone replacement material in the collagen matrix improves the compressibility of the shaped body, in particular in the regions, in which only little or no bone replacement material at all is provided. The compressibility of the shaped body facilitates the insertion of the shaped body into the wound cavity (simple pressing together between thumb and index finger, then insertion into the wound cavity) and allows better and gentler close fitting against the wall of the wound cavity, for example the alveolus, without additional injuries occurring in the process. This results in improved wound healing and bone regeneration or preservation.

The shaped body can be adapted to the respective bone defect location with low outlay during the treatment sitting by the dentist/doctor. On the one hand, this is achieved by the compressibility. On the other hand, the shaped body can, in particular, also be cut. Cutting the shaped body may preferably take place by means of a conventional cutting tool, such as, for example, a knife or a scalpel. A special tool for this can be dispensed with. The simple and good adaptability of the shaped body to the bone defect location also ultimately leads to very good wound healing, bone regeneration and preservation.

Because of the bone replacement material concentration, which is at a maximum at the edge face and decreases inwardly, the bone replacement material is introduced in a targeted manner above all where its mechanically supporting function is particularly required, namely, in particular in the opening region of a bone defect location.

Overall, the shaped body according to the invention provides a “regenerative space”, because of which the physiological wound healing is promoted and a loss of bone mass is avoided.

A configuration is favorable, in which the shaped body at a compression by up to 40% of its starting volume has a compressive modulus of at most 1.0 MPa, in particular at most 0.5 MPa, preferably at most 0.25 MPa. The compression thus takes place to up to 60% of the starting volume of the shaped body. Therefore, the shaped body has a virtually equally favorable compression behavior to a comparative shaped body consisting completely of collagen material. For a compression of the shaped body by up to 30% of its starting volume, in other words to up to 70% of its starting volume, in particular at most a compressive force is required, which is in particular at most twice as great, preferably even only substantially the same, as in the comparative shaped body consisting completely of collagen material. For a compression of the shaped body by up to 40% of its starting volume, in particular, a compressive force of at most 50 N, in particular of at most 25 N, preferably of at most 10 N is required. Moreover, the compressive deformation of the shaped body is advantageously plastic. The compressive deformation thus reverses again after insertion in the bone defect location, at least as far as the local conditions allow and fits closely against the walls of the bone defect location in the process. As a result, cavities are substantially avoided, which has a favorable effect on the wound healing and the bone regeneration.

According to a further favorable configuration, the maximum value of the local bone replacement material proportion at the edge face based on a part volume of 30 mm³ is between 10% and 90%. In particular, the maximum local bone replacement material proportion may be present at an area proportion based on the total edge face of between 10% and 100%. The quantity of the bone replacement material introduced in the respective application can also thus be individually adapted to the case-specific requirements.

A further configuration is favorable, in which the reduction in the bone replacement material proportion in the BRM zone takes place continuously or with at least a discrete graduation, preferably with a plurality of discrete graduations, or continuously in regions and graduated in regions, in particular up to a minimum local bone replacement material proportion between 1% and 40%. A very precise adaptation to the local requirements can also take place by means of these design measures.

According to a further advantageous configuration, the shaped body has an elongate basic shape with a longitudinal axis and with an end limiting face arranged perpendicular to the longitudinal axis. The end limiting face, which may, during the application, in particular be the upper end face, i.e. facing the opening of the bone defect location, is then that edge face, proceeding from which the local bone replacement material proportion decreases. An elongate shaped body of this type is very suitable as an alveolar filling body, i.e. for use for treating the wound cavity after a tooth extraction. In this application, it is advantageous if the bone replacement material is mainly introduced at the top at the opening of the alveolus and the bone replacement material concentration decreases toward the alveolus base.

According to another favorable configuration, the shaped body has an elongate basic shape with a longitudinal axis and with a lateral limiting face extending substantially in the direction of the longitudinal axis or, in particular, also at an acute angle of inclination to the longitudinal axis. The lateral limiting face is then that edge face, proceeding from which the local bone replacement material proportion decreases. This configuration is particularly suitable for applications, in which the maximum bone replacement material concentration is not required at the top or bottom at a bone defect location to be treated, but at a side wall of the bone defect location. The latter may, for example, be a bone defect, which remains after the removal of a malformation, in particular in the mouth-jaw-face region, such as, for example, a cyst, an ulcer or a tumor. A bone defect of this type differs with respect to its structure and its orientation from a tooth alveolus, so a maximum concentration of the bone replacement material may be advantageous on the side wall of the shaped body in an application of this type.

A further configuration is favorable, in which the BRM zone has, along its extent direction into the interior, based on a BRM starting face of the edge face provided with bone replacement material, perpendicular to the extent direction, cross sectional faces, which are larger or smaller than the BRM starting face or the same size as the BRM starting face. The BRM zone can therefore, in particular widen, narrow or remain the same size within the shaped body. The most suitable configuration in this regard can readily be selected in accordance with the specific requirements. Configurations are particularly favorable, the BRM zone of which narrows along its extent direction into the interior. In particular, a configuration with a BRM zone in a funnel-like shape, preferably in the shape of a part of a rotational hyperboloid, produces, for the alveolar application, a particularly advantageous combination of compressibility in the lower part and an upwardly increasing support of the alveolus by bone replacement material. This also applies to an alternative configuration with a drop-shaped BRM zone, which also narrows at least a certain amount in the extent direction.

According to a further favorable configuration, the BRM zone substantially has a drop-like shape or substantially a funnel-like shape, in particular a conical or truncated cone shape, or at least partly the shape of a part of a rotational hyperboloid or substantially a toroidal shape. By means of these various configurations, practically all conceivable applications can be covered very well. In the rotational hyperboloid configuration, the BRM zone in particular has substantially the shape of the upper half of a rotational hyperboloid. Moreover, the design of the BRM zone in this configuration may deviate slightly from the mathematically precise shape of a (part of the) rotational hyperboloid, in particular at the upper and lower edge faces of the BRM zone. As already mentioned, the configurations with a BRM zone in the form of a part of a rotational hyperboloid and with a drop-shaped BRM zone are particularly suitable for an alveolar use.

According to a further favorable configuration, the collagen material and the bone replacement material recognizably differ from one another, in particular at least one material from the group of the collagen material and the bone replacement material being dyed. The treating doctor can then better recognize the layer of the bone replacement material non-homogeneously embedded in the shaped body. This facilitates the handling, in particular the correct cutting of the shaped body (if cutting of this type should be necessary) and the correctly oriented insertion of the shaped body in the bone defect location.

A further configuration is favorable, in which the bone replacement material is prepared native bone material, in particular purified spongy bone material, or synthetic bone replacement material, in particular tricalcium phosphate granulate, hydroxylapatite granulate, resorbable bioceramic granulate, such as, for example, bioglass granulate, biphasic bone replacement material granulate or multiphasic bone replacement material granulate. Biphasic bone replacement material granulate is a composite of two synthetic bone replacement materials and multiphasic bone replacement material is a composite of more than two synthetic bone replacement materials. The preparations of the bone replacement material granulates may also contain further bioresorbable polymers, such as, for example, binders in the form of polylactide. The bone replacement material is in each case, in particular grainy and has a preferred particle diameter between 0.05 mm and 3 mm, in particular between 0.1 mm and 2 mm, preferably between 0.2 mm and 1 mm. Bone replacement material according to these specifications in each case has a very good mechanically stabilizing effect and promotes the bone formation in the bone defect location during the wound healing process.

A variant is favorable, in which the collagen material consists at least partly of a porous collagen, for example of lyophilized, dried collagen or collagen produced by means of felting, preferably of reconstituted type 1 collagen. The collagen material is advantageously of equine origin. It is furthermore preferably provided that the collagen material has a density of 1 to 25 mg/cm³, preferably of 5 to 12 mg/cm³. Collagen with these density values can be produced particularly well. Collagen is very well tolerable as a bioresorbable material and is used for haemostasis, for filling bones and tissue defects and for covering wounds. Collagen assists the haemostasis, in that thrombocytes aggregate at the collagen fibrils and form a coagulum. The collagen is completely resorbed during the course of wound healing by the effect of immigrated macrophages and collagenase particular to the body. A variant, in which, at least partly, particularly hydrophilic, easily wetted collagen material is used, is also favorable. This more hydrophilic collagen material can be produced by a special selection of raw material or the addition of hydrophilic substances.

A further configuration is favorable, in which the shaped body has a substantially elongate basic shape with a longitudinal axis, the extent in the direction of the longitudinal axis being between 0.4 cm and 3 cm and the maximum extent perpendicular to the longitudinal axis being between 0.3 cm and 2.9 cm. Elongate shaped bodies with these dimensions are very suitable for introduction into an alveolar space. An additional cut can then be dispensed with in many cases.

According to another favorable configuration, the shaped body has a substantially cube-shaped basic shape, the extent in the direction of the three cube axes in each case being between 0.3 cm and 3 cm. Shaped bodies of this type are particularly suitable for filling cavities within the bone defect locations, which are in some circumstances difficult to access from outside and into which another (functional) body, such as, for example, an implant, has been introduced.

A further configuration, in which the shaped body is a composite body with a first part body made of the composite material with bone replacement material incorporated, distributed non-homogeneously, in a collagen matrix and with a second part body rigidly connected to the first part body and configured as a covering membrane, is favorable. A membrane face of the second part body is greater than a base face of the first part body. The second part body is placed on the first part body in such a way that the second part body projects laterally everywhere beyond the base face of the first part body. The arrangement of a laterally projecting covering membrane on a base body (=first part body) is already known from DE 10 2008 010 893 B3. The advantages and configurations described there with regard to the covering membrane are produced equally in a combination of a base body, which is produced from the composite material with bone replacement material incorporated non-homogeneously distributed in a collagen matrix, with the covering membrane. In particular, the covering membrane covers the alveolus with its edge region projecting laterally beyond the base body and thus forms an effective barrier against uncontrolled connective and epithelial tissue proliferation into the alveolus. As bone tissue proliferates substantially more slowly in comparison to the connective and epithelial tissue, without the covering membrane the danger would exist of the connective and epithelial tissue filling up the alveolar space more quickly and therefore impairing the bone growth.

According to a further preferred configuration, the composite material contains a wound-healing bioactive component promoting the angiogenesis or promoting the bone growth. This bioactive component is preferably a native isolated or biotechnologically obtained protein, namely BMP-2 (=Bone Morphogenic Protein 2). This also includes inter alia TGF beta (TGF=Transforming Growth Factor) or the like. The angiogenesis-promoting factors, such as, for example, natively isolated or biotechnologically produced FGF-1 (Fibroblast Growth Factor 1), VEGF-A (Vascular Endothelial Growth Factor A) or the like, are also included in this.

According to another also favorable configuration, the composite material contains an antimicrobial or antibiotically acting component. This antimicrobial component contributes to preventing and/or combating infections. This is preferably a locally tolerable antiseptic, such as, for example, polyhexanide, octenidine, silver ions, iodine derivatives, chlorhexidine, triclosan or the like. Likewise, an antibiotically acting substance suitable for local application, such as, for example, gentamicin, metronidazole, vancomycin, clindamycin or the like, can also be used.

Further features, advantages and details of the invention emerge from the following description of embodiments with the aid of the figures of the drawings

### BRIEF DESCRIPTION OF THE DRAWINGS

FIGS. 1 to 13 show embodiments of a shaped body with collagen-containing composite material for introduction into a bone defect location,

FIG. 14 shows a graph with measurement curves for compressive force courses depending on the compression for various collagen-containing shaped bodies, and

FIG. 15 shows an embodiment for the use of the shaped bodies according to FIGS. 1 to 12 for filling cavities between an implant and the alveolus walls.

### DESCRIPTION OF THE PREFERRED EMBODIMENTS

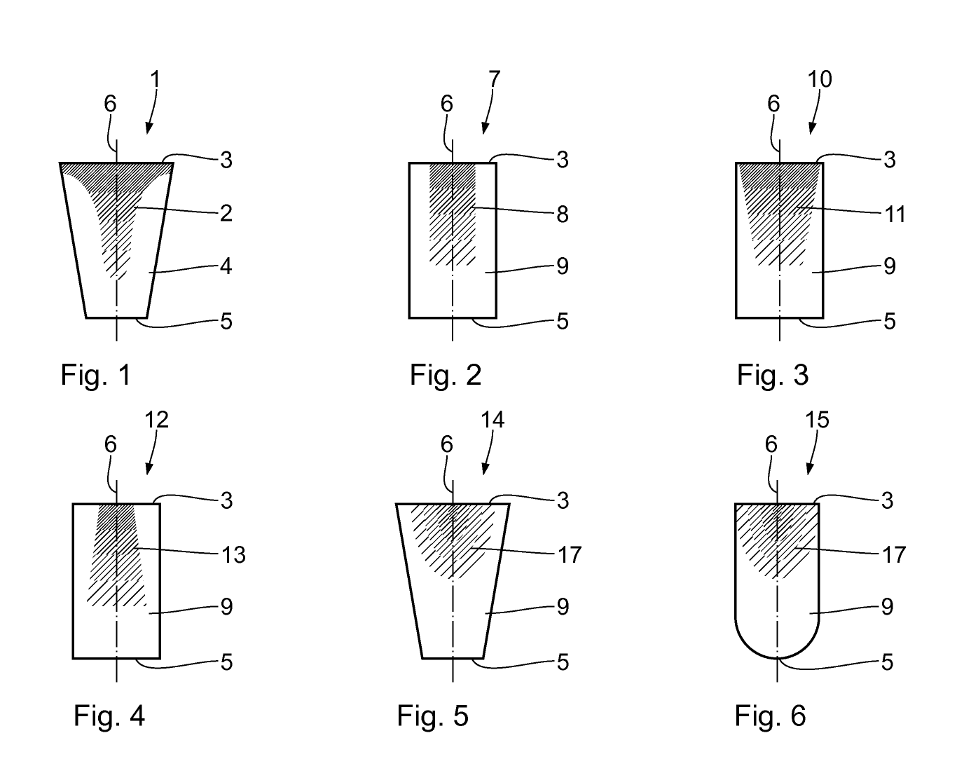

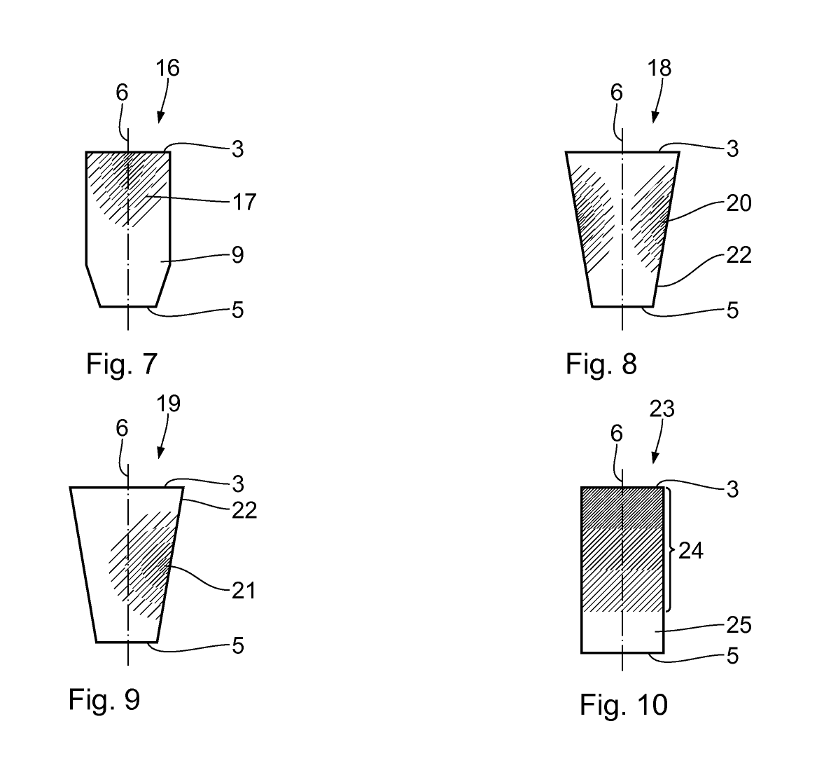

Parts corresponding to one another are provided with the same reference numerals in FIGS. 1 to 15. Details of the embodiments described in more detail below may be an invention per se or be parts of an inventive subject.

FIG. 1 shows a shaped body 1 made of a composite material in a longitudinal sectional view. The shaped body 1 has a truncated cone shape. The composite material contains a matrix made of collagen material, in which, in regions, namely within a BRM zone 2, bone replacement material is embedded. The BRM zone 2 substantially has the shape of the upper half of a rotational hyperboloid, the BRM zone 2 differing at the upper and lower end from the mathematically precise hyperboloid shape.

The bone replacement material is non-homogeneously distributed within the BRM zone 2. The maximum bone replacement material concentration is at an upper edge face 3, which, in the embodiment shown of the elongate shaped body 1, is the upper end limiting face. The BRM zone 2 extends proceeding from the edge face 3 into the interior of the shaped body 1, the concentration of the bone replacement material decreasing substantially continuously inwardly proceeding from the edge face 3. A stepped course is indicated in FIG. 1 purely for better illustration. In fact, the concentration reduction of the bone replacement material runs in a more fluent manner, however. The BRM zone 2 does not fill the entire shaped body 1. There is also a bone replacement material-free zone 4, which also, in particular, adjoins a lower edge face 5 opposing the upper edge face 3.

The shaped body 1 has a substantially rotationally symmetrical structure in relation to a longitudinal axis 6. It has a longitudinal extent in the direction of the longitudinal axis of 1.6 cm. At the upper edge face 3, its extent perpendicular to the longitudinal axis 6, in other words the diameter, is about 1 cm.

The collagen material in the embodiment has a density of 11.2 mg/cm³. The global collagen proportion of the entire shaped body 1 is about 80%. The local collagen proportion in the bone replacement material-free zone 4, which, in this embodiment, takes up about 75% of the total volume of the shaped body 1, is naturally particularly high, in particular approximately 100%. However, collagen material is also present everywhere in the BRM zone 2. The local collagen proportion based on any desired part volume of 30 mm³ is at least 10% everywhere. This also applies, in particular, in the zone adjoining the upper edge face 3, within which the concentration of the bone replacement material is maximal.

The BRM zone 2 contains a total of 0.1 g of non-homogeneously distributed bone replacement material in the form of tricalcium phosphate granulate. The BRM zone 2 makes up about 25% of the total volume of the shaped body 1. The local bone replacement material proportion based on a part volume of 30 mm³ in size, at about 40%, has its maximum value at the upper end face 3.

The shaped body 1 is intended, in particular, for insertion in a bone defect location, in particular in a tooth pocket (=alveolus), which is formed, for example, after a tooth extraction. The shaped body 1 is thus an alveolus filling body, which is preferably to be inserted into the alveolus such that the lower edge face 5 rests on the alveolar base.

The insertion of the shaped body 1 into the alveolus is facilitated in that the shaped body 1 is compressible. The good compressibility results from the particularly advantageous structure in this regard of the shaped body 1 with the soft, plastically deformable collagen matrix, in which the mechanically more rigid and less well compressible bone replacement material is embedded non-homogeneously distributed and with a local maximum concentration at an edge face of the shaped body 1.

The two material components of the shaped body 1 each contribute in their way to a particularly favorable healing course and to very good bone regeneration. It has been shown that no bone replacement material is required for favorable bone regeneration of this type precisely in the region of the alveolar base. The mechanically stabilizing effect thereof, in contrast to the alveolar opening, is not required at the alveolar base. The collagen material present there is completely sufficient for bone regeneration. The collagen material, compared with the bone replacement material, is resorbed substantially more quickly. The wound healing and the bone regeneration accelerate as a result. On the other hand, the stabilizing effect of the bone replacement material at the alveolar opening is certainly desired and sensible.

FIG. 2 shows a further embodiment of a shaped body 7, which is also manufactured from the collagen-containing composite material. The shaped body 7 also contains a BRM zone 8 with a non-homogeneously distributed embedding of bone replacement material within the collagen matrix. The shaped body 7, like the BRM zone 8, has a cylindrical shape. The BRM zone 8 is surrounded by a bone replacement material-free zone 9. The maximum local bone replacement material proportion of the BRM zone 8 in turn adjoins the upper edge face 3, with the BRM zone 8 in the shaped body 7, unlike the shaped body 1, not extending over the entire face of the edge face 3, but only over a central region of the edge face 3. This central region is about 60% of the entire edge face 3. The proportion of the bone replacement material reduces proceeding from the edge face 3 within the shaped body 7 in the direction of the longitudinal axis 6. The extent of the BRM zone 8 perpendicular to the longitudinal axis 6 remains substantially the same within the shaped body 7.

FIG. 3 shows a further embodiment of a shaped body 10 with bone replacement material embedded non-homogeneously in a collagen matrix. The shaped body 10 is cylindrical. A funnel-shaped or truncated cone-shaped BRM zone 11 in this embodiment extends proceeding from the edge face 3 with the maximum bone replacement material concentration into the interior of the shaped body 10, the maximum extent of the BRM zone 11 perpendicular to the longitudinal axis 6 being provided at the upper edge face 3. The extent of the BRM zone 11 perpendicular to the longitudinal axis 6 decreases within the shaped body 10 like the proportion of bone replacement material.

An embodiment of a similar cylindrical shaped body 12 is shown in FIG. 4. It also contains a funnel-shaped or truncated cone-shaped BRM zone 13, the opening angle of the truncated cone-shaped BRM zone 13 being oriented precisely in the opposing direction to in the BRM zone 11 of the shaped body 10. Accordingly, the BRM zone 13 in this embodiment has the minimum extent perpendicular to the longitudinal axis 6 at the upper edge face 3. The extent of the BRM zone 13 perpendicular to the longitudinal axis 6 increases within the shaped body 12. The proportion of bone replacement material decreases, on the other hand, within the shaped body 12.

FIGS. 5 to 7 show further embodiments of shaped bodies 14 to 16 made of collagen-containing composite material. These shaped bodies 14 to 16 in each case contain a drop-shaped BRM zone 17 with a maximum concentration of the bone replacement material adjoining the upper edge face 3 in the region of the longitudinal axis 6. Proceeding from this region with a maximum concentration of bone replacement material, the proportion of bone replacement material decreases both parallel and perpendicular to the direction of the longitudinal axis 6. The shaped body 14 is truncated cone-shaped, whereas the shaped bodies 15 and 16 have a cylindrical basic shape, which, at the lower end opposing the upper edge face 3 in the shaped body 15, passes into a hemispherical shape and, in the shaped body 16, into a truncated cone shape.

The further embodiments in FIGS. 8 and 9 of shaped bodies 18 and 19 also consist of the collagen-containing composite material and accordingly have a BRM zone 20 or 21. The shaped bodies 18 and 19 in each case have a truncated cone shape. In contrast to the previous embodiments, the BRM zones 20 and 21 do not adjoin the upper end face 3, but the truncated cone-shaped lateral face 22, which is also an edge face of the shaped bodies 18 and 19. The BRM zones 20 and 21 in turn have, adjoining the lateral face 22, the region with the maximum concentration of the bone replacement material. Proceeding from this, the concentration of the bone replacement material also decreases at least inwardly. The shaped body 18 is rotationally symmetrical in relation to the longitudinal axis 6. The BRM zone 20 has a substantially toroidal shape. Compared to this, the BRM zone 21 is not rotationally symmetrical. In the shaped body 19, the BRM zone 21 is only configured as a drop-shaped zone at one location of the lateral face 22.

FIG. 10 shows a further embodiment of a collagen-containing shaped body 23, which also comprises a BRM zone 24. The shaped body 23 is cylindrical, just like the BRM zone 24. The BRM zone 24 in this embodiment over the entire area adjoins the upper edge face 3 and has the maximum bone replacement material concentration in this region. In contrast to the previous embodiments, no continuous reduction in the bone replacement material concentration is provided in the BRM zone 24, but a stepped reduction therein. Unlike the previous embodiments, the stepped course of the bone replacement material concentration shown in FIG. 10 in this embodiment thus corresponds to the actual facts. The reduction takes place proceeding from the edge face 3 inwardly and in the direction of the longitudinal axis 6. The BRM zone 24, in the embodiment shown, comprises three part zones with, in each case, a bone replacement material concentration which is uniform within the part zone but different from one another. The BRM zone 24 at its lower end passes, i.e. at the end opposing the edge face 3, with a last graduation step from the lowermost part zone with the lowest bone replacement material concentration, into a bone replacement material-free zone 25.

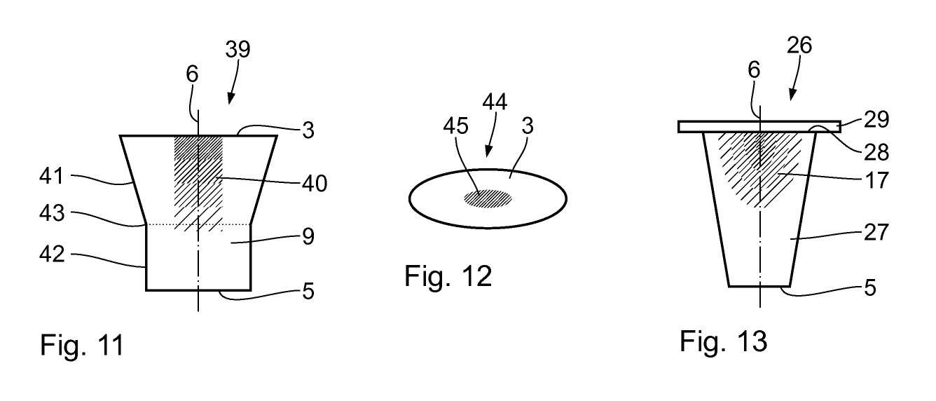

FIG. 11 shows a further embodiment of a shaped body 39 made of collagen-containing composite material. It comprises a cylindrical BRM zone 40 with a non-homogeneously distributed embedding of bone replacement material within the collagen matrix. The BRM zone 40 is similarly constructed to the BRM zone 8 of the embodiment according to FIG. 2. The shaped body 39, however, has a slightly different external contour. It is composed of an upper truncated cone-shaped part portion 41 and a lower cylindrical part portion 42. The two part portions 41 and 42 adjoin one another at an abutting face 43. The BRM zone 40 in the embodiment shown is substantially arranged within the upper part portion.

FIG. 12 shows a further embodiment of a collagen-containing shaped body 44. No longitudinal section is shown here, but a plan view of the upper edge face 3 of the shaped body 44. The shaped body 44 also has a BRM zone 45 with a non-homogeneously distributed embedding of bone replacement material within the collagen matrix. In contrast to the previous embodiments, the shaped body, however, does not have a circular, but an oval cross sectional face. Its longitudinal section, on the other hand, looks just as in the embodiment according to FIG. 11. Basically, the other embodiments of shaped bodies could also have an oval cross sectional face instead of the round one perpendicular to the centre longitudinal axis 6.

The further embodiment of a shaped body 26 shown in FIG. 13 is similar to the shaped body 14 according to FIG. 5. The shaped body 26 has a truncated cone-shaped base body 27, on the upper edge face 28 of which is arranged a covering membrane 29. The base body 27 substantially corresponds to the shaped body 14 according to FIG. 5, wherein no restriction is to be seen here. In principle, every other one of the shaped bodies 1, 7, 10, 12, 15, 16, 18, 19, 23, 39 and 44 shown in FIGS. 1 to 12 could be provided with a cover comparable to the covering membrane 29. The covering membrane 29 projects over the edge face 28 everywhere. It is rigidly connected to the base body 27. The mode of action and particular configurations of the covering membrane 29 are described in DE 10 2008 010 893 B3.

The shaped bodies 1, 7, 10, 12, 14, 15, 16, 18, 19, 23, 26, 39 and 44 shown in FIGS. 1 to 13 are in each case produced with the aid of the collagen-containing composite material. In each case, they have a proportion of bone replacement material. Nevertheless, they are compressible and can therefore be easily inserted into a bone defect location, without in the process causing subsequent injuries to the walls of the bone defect location.

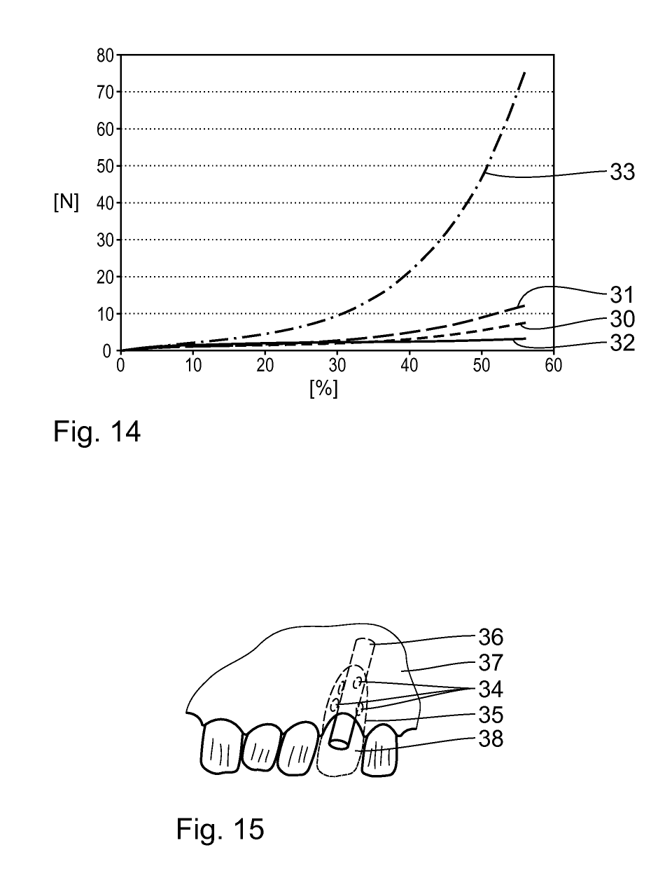

The good compressibility is documented with the aid of the measurement curves shown in FIG. 14. The measurements were carried out with the aid of two shaped bodies 14 according to FIG. 5. In the first shaped body 14, the BRM zone 17 contained a total of 0.1 g bone replacement material (see measurement curve 30 with the line with short dashes), whereas in the second shaped body 14, the BRM zone 17 contained a total of 0.2 g bone replacement material (see measurement curve 31 with the line with long dashes). In the graph according to FIG. 14, a first comparative measurement curve 32 (continuous line) is entered for a first comparative shaped body consisting exclusively of collagen material. A second comparative measurement curve 33 (dash-dot line) was determined for a second comparative shaped body, in which a total quantity of 0.3 g bone replacement material was embedded, homogeneously distributed. The second comparative measurement body thus did not contain, in particular, a bone replacement material-free zone. Its bone replacement material concentration was substantially the same size everywhere.

The course of the compressive force over the compression based on the starting volume, by which the relevant shaped body was compressed, is plotted in the graph according to FIG. 14. It can be inferred from the measurement graph that the two investigated shaped bodies 14 (measurement curves 30 and 31) up to a 30% compression have an almost identical compression behavior to the first comparative shaped body (measurement curve 32) consisting exclusively of collagen material. In comparison to this, in the second comparative shaped body with homogeneously embedded bone replacement material (measurement curve 33), a noticeably higher compressive force is already required at a compression by 7%. Because of the non-homogeneously distributed embedding of the bone replacement material, the compression behavior in the shaped bodies 14 is not substantially impaired up to a compression by about 40%. In contrast to this, the second comparative shaped body with homogeneously incorporated bone replacement material has a significantly worse compressibility.

To this extent, the shaped body 14 as well as the other shaped bodies 1, 7, 10, 12, 15, 16, 18, 19, 23, 26, 39 and 44 shown in FIGS. 1 to 13, compared with a shaped body with homogeneously incorporated bone replacement material, also provides significant advantages in handling, in particular during insertion into the bone defect location as well as during the close fitting against the wall of the bone defect location. The latter also favors particularly advantageous wound healing and bone regeneration.

The shaped bodies 1, 7, 10, 12, 14, 15, 16, 18, 19, 23, 26, 39 and 44 may be approximately adapted to the size of the bone defect location with respect to their respective external dimensions. In these configurations, a single one of the shaped bodies 1, 7, 10, 12, 14, 15, 16, 18, 19, 23, 26, 39 and 44 is in each case inserted in the bone defect location.

However, there is also another application shown in FIG. 15 with shaped bodies 34 made of collagen-containing composite material with bone replacement material embedded non-homogeneously distributed. The shaped bodies 34 are (significantly) smaller than the bone defect location 35 (=alveolus). In this application, immediately after the tooth extraction, a pin-shaped implant 36 is inserted into the remaining jaw bone 37. An implant crown 38 is placed on the implant 36 after the healing process. Cavities remain between the implant 36 and the walls of the bone defect location 35 and are filled with shaped bodies 34 to promote the wound healing process and the bone growth, in particular the ingrowth of the implant 36. The shaped bodies 34 may have a substantially similar or the same structure as the shaped bodies 1, 7, 10, 12, 14, 15, 16, 18, 19, 23, 26, 39 and 44. Alternatively, other external contours with, for example, a cube-shaped or parallelepiped shape are also possible, however. Advantageously, the shaped bodies 34 also have the favorable compressibility, so they can be inserted well into the cavities, which are only accessible with difficulty under some circumstances. They expand again there and substantially completely fill the cavities, so the close fitting against the walls of the wound cavity (=bone defect location 35) favorable for the healing and bone growth process is also provided.

What is claimed is:

1. A shaped body for introduction into an alveolar space or into another bone defect location (35), wherein
a) a composite material with at least a first material component in the form of a collagen material and a second material component in the form of a bone replacement material is provided for the shaped body (1; 7; 10; 12; 14; 15; 16; 18; 19; 23; 26; 34; 39; 44),
b) the collagen material forms a matrix, in which the bone replacement material is embedded, distributed non-homogeneously, so that a local collagen proportion based on any desired part volume of 30 mm³ is at least 10% everywhere and a global collagen proportion based on the total volume is in the range between 30% and 95%,
c) the shaped body (1; 7; 10; 12; 14; 15; 16; 18; 19; 23; 26; 34; 39; 44) is compressible, and
d) a local bone replacement material proportion based on a part volume of 30 mm³ has its maximum value at an edge face (3; 22) and decreases inwardly proceeding from the edge face (3; 22), a BRM zone (2; 8; 11; 13; 17; 20; 21; 24; 40; 45) provided with bone replacement material extending into the interior proceeding from the edge face (3; 22).

2. A shaped body according to claim 1, wherein the shaped body (1; 7; 10; 12; 14; 15; 16; 18; 19; 23; 26; 34; 39; 44) at a compression by up to 40% of its starting volume, has a compressive modulus of at most 1.0 MPa.

3. A shaped body according to claim 1, wherein the maximum value of the local bone replacement material proportion based on a part volume of 30 mm³ at the edge face (3; 22) is between 10% and 90%.

4. A shaped body according to claim 1, wherein the maximum local bone replacement material proportion is present at an area proportion based on the total edge face (3; 22) of between 10% and 100%.

5. A shaped body according to claim 1, wherein the reduction in the bone replacement material proportion in the BRM zone (2; 8; 11; 13; 17; 20; 21; 24; 40; 45) takes place one of continuously and with at least a discrete graduation, and continuously in regions and graduated in regions.

6. A shaped body according to claim 5, wherein the reduction in the bone replacement material proportion in the BRM zone (2; 8; 11; 13; 17; 20; 21; 24; 40; 45) takes place with a plurality of discrete graduations.

7. A shaped body according to claim 5, wherein the reduction in the bone replacement material proportion in the BRM zone (2; 8; 11; 13; 17; 20; 21; 24; 40; 45) takes place up to a minimum local bone replacement material proportion of between 1% and 40%.

8. A shaped body according to claim 1, wherein an elongate basic shape with a longitudinal axis (6) and with an end limiting face (3) arranged perpendicular to the longitudinal axis (6) is provided, and the end limiting face is the edge face (3), proceeding from which the local bone replacement material proportion decreases.

9. A shaped body according to claim 1, wherein an elongate basic shape with a longitudinal axis (6) and with a lateral limiting face (22) extending substantially in the direction of the longitudinal axis (6) is provided, and the lateral limiting face is the edge face (22), proceeding from which the local bone replacement material proportion decreases.

10. A shaped body according to claim 1, wherein the BRM zone (2; 8; 11; 13; 17; 20; 21; 24; 40; 45), along its extent direction into the interior, in relation to a BRM starting face of the edge face (3; 22) provided with bone replacement material, perpendicular to the extent direction, has cross sectional faces, which are greater or smaller than the BRM starting face or the same size as the BRM starting face.

11. A shaped body according to claim 1, wherein the BRM zone (2; 8; 11; 13; 17; 20; 21; 24; 40; 45) has substantially a drop-like or funnel-like shape or at least partially the shape of a part of a rotational hyperboloid or a substantially toroidal shape.

12. A shaped body according to claim 1, wherein the collagen material and the bone replacement material recognizably differ from one another.

13. A shaped body according to claim 1, wherein at least one material from the group of the collagen material and the bone replacement material is dyed.

14. A shaped body according to claim 1, wherein the bone replacement material is prepared native bone material, or synthetic bone replacement material.

15. A shaped body according to claim 14, wherein the bone replacement material is purified spongy bone material.

16. A shaped body according to claim 14, wherein the bone replacement material is one of the group of tricalcium phosphate granulate, hydroxylapatite granulate, resorbable bioceramic granulate, biphasic and multiphasic bone replacement material granulate.

17. A shaped body according to claim 14, wherein the bone replacement material is grainy.

18. A shaped body according to claim 17, wherein the bone replacement material has a preferred particle diameter of between 0.05 mm and 3 mm.

19. A shaped body according to claim 1, wherein the shaped body (1; 7; 10; 12; 14; 15; 16; 18; 19; 23; 26; 34; 39; 44) has a substantially elongate basic shape with a longitudinal axis (6), the extent in the direction of the longitudinal axis (6) being between 0.4 cm and 3 cm and the maximum extent perpendicular to the longitudinal axis being between 0.3 cm and 2.9 cm.

20. A shaped body according to claim 1, wherein the shaped body has a substantially cube-shaped basic shape, the extent in the direction of the three cube axes being between 0.3 cm and 3 cm, in each case.
